(19) United States
(12) Patent Application Publication
(10) Pub. No.: US 2008/0213719 A1
(43) Pub. Date: Sep. 4, 2008

(54) Temperature Modified Oral Cleaning Device

(76) Inventors: Martin S. Giniger, Beaverton, OR (US); Rowland Hanson, Kirkland, WA (US)

Correspondence Address: BERENBAUM, WEINSHIENK & EASON, P.C, 370 17TH STREET, SUITE 4800, DENVER, CO, 80202, US

(21) Appl. No.: 12/014,021

(22) Filed: Jan. 14, 2008

Related U.S. Application Data
(60) Provisional application No. 60/884,620, filed on Jan. 12, 2007.

(51) Int. Cl. A61C 17/00; A61F 7/00; A61C 15/00; A46B 9/04

(52) U.S. Cl. 433/32; 607/96; 433/216; 15/167.1

(57) ABSTRACT

A temperature modified oral care device including a base; an electrical circuit disposed at least partially in said base; and, a heating element connected to the electrical circuit.

### CROSS REFERENCE TO RELATED APPLICATION

This application claims the benefit of priority under 35 U.S.C. § 119(e) from U.S. Provisional Patent Application No. 60/884,620, entitled “Rapid Heating Oral Cleaning Device”, filed Jan. 12, 2007, which is incorporated herein in its entirety by this reference.

### BACKGROUND

The present disclosure is based on the general understanding that heat may enhance a cleaning process. That understanding derives from the effects of heat on either chemical or physical processes, or both. Here, the principle of enhancement of the cleaning process by heat is applied to an oral care cleaning process.

It is known that conducting a chemical reaction at a higher temperature may generally involve the delivery of more energy into the system which may increase the reaction rate by causing more collisions between particles, as explained by collision theory. Additionally, heat and/or energy may increase the rate of reaction for the reason that more of the colliding particles will have the necessary, increased activation energy to result in more successful collisions, particularly when the success of a collision is measured in the resultant bonds formed between reactants.

The influence of temperature on reactions may be described by the Arrhenius equation. At higher temperatures, the probability that two molecules will collide is higher; this higher collision rate results in a higher kinetic energy, which has an effect on the activation energy of the reaction; the activation energy is the amount of energy required to ensure that a reaction happens.

The Arrhenius equation is expressed as: k=A*exp(−Ea/R*T); where k is the rate coefficient, A is a constant, Ea is the activation energy, R is the universal gas constant, and T is the temperature (in degrees Kelvin); and R has the value of 8.314×10-3 kJ mol-1K-1. As a rough rule of thumb, reaction rates for many reactions double or triple for every 10 degrees Celsius increase in temperature.

Rapid heating is a generally known concept available in a variety of forms. These may include implementations such as those available from the ColdHeat Company of Washington, USA, or from Hyperion Innovations, Inc., Bellevue, Wash., USA, inter alia. An implementation hereof may include a resistance heating, as where one or more elements of a high resistance material may be disposed in or be connected to an electrical circuit, the high resistance creating a great deal of heat quickly when exposed to the flow of electricity. The ColdHeat company uses graphite, a composition containing graphite, germanium or silicon or an athalite composition. Typically and particularly, this composition includes a high carbon content, and thus has a high resistance, although it is electrically conductive, or semi-conductive and thus may be disposed in an electrical circuit. The graphite or athalite (or other) element may heat up quickly when disposed in a live circuit, and conversely may cools quickly when current flow is halted. Induction heating, which involves electromagnetically inducing a current in a conductive receiver without contact, may also be used, together or in the alternative.

### SUMMARY

The present improvements include heated oral care devices, such as toothbrushes, tooth polishers and/or tooth whitening trays. A rapid heating oral care device is described as well, as are such devices which may provide rapid cooling. A heating element using graphite, a composition containing graphite, germanium or silicon or an athalite composition is also described.

### BRIEF DESCRIPTION OF DRAWINGS

In the drawings:

FIG. 1 provides an isometric view of a heated toothbrush according hereto;

FIG. 2 provides an isometric view of a heated toothbrush according hereto; and,

FIG. 3 provides an isometric view of a heated toothbrush according hereto.

### DETAILED DESCRIPTION

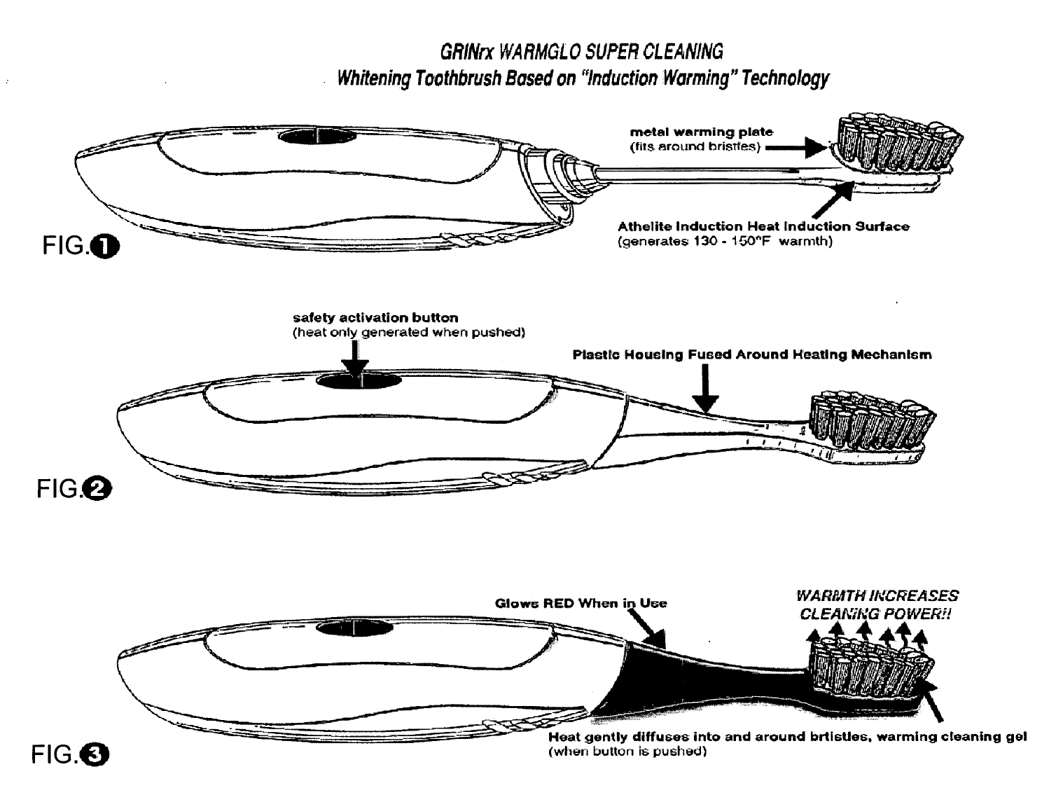

The present improvements include heated oral care devices, such as toothbrushes, tooth polishers and/or tooth whitening trays. In the drawing Figs., a heated toothbrush is shown. In FIG. 1, a metal, metallic or other heat conductive plate is shown holding the bristles. The metal plate may be in connection with an athalite heating surface which may be connected, via an elongated connection to the base or handle, to an electrical circuit in the device. The electrical circuit may include wires or other conductive components connected between the heating element at the brush head to batteries or another power source in the base/handle, and a power switch, as shown, for example, in FIG. 2. Note, the button activation may be considered a safety feature in that heat would only be generated if the safety button were pushed in this embodiment.

The device may have a plastic housing, or a housing of another material, particularly suited to be heat resistant.

As shown in FIG. 3, heat may then emanate from the heating surface to, around and through the bristles to provide heat to the bristle tip area so that heat may reach the cleaning surface during use, as in use in cleaning teeth or other oral features (tongue, palate, etc.). A toothpaste, gel or other dentifrice could also be used, and warmed by the emanating heat, thereby enhancing its effectiveness.

Also shown as an option in FIG. 3 is a lighted feature which may light up when the current is activated and the heating element is heating up. Red or some other color could also/alternatively be used.

A rapid heating oral care device may also be provided as well as alternative rapid cooling devices. A heating element using a graphite composition or other high carbon or similar composition may provide either or both rapid heating and/or cooling due to the high resistance values thereof. Other potentially useful compositions may include a composition containing graphite, germanium or silicon or an athalite composition, or like compositions providing for high resistance, and thus high, rapid heat generation. Low thermal conductivity might be another/alternative property of desirable compositions hereof or useful herewith.

Features of many such implementations may include, but not be limited to: an athalite alloy induction heating/cooling system; a handle switch feature that, when pushed up, turns the unit on, and when pushed down, turns the unit off; when the unit is turned on, the bristle pad may instantly heat; the bristle pad may radiate heat into the bristles and applied toothpaste; the toothpaste may be constantly heated for over 3 minutes; the toothpaste temperature may increase 10° C., at which point, according to the Arrhenius equation, the cleaning efficiency may be doubled; cleaning may be effected twice as quickly, which may be more convenient and may increase the level of oral hygiene. According to the American Dental Association, the typical adult brushes for less than the ADA recommended time of 2-3 minutes: the average adult brushes for only 51 seconds and the average 11 year old child for 13 seconds; and, better stain removal may also be effected and may be desirable in that the more stains are removed, the whiter-looking the teeth become.

As introduced above, conducting a chemical reaction, such as a cleaning operation, at a higher temperature may deliver more energy into the system and increase the reaction rate by causing more collisions between particles, as explained by collision theory, and increasing the activation energy, resulting in more successful collisions (when bonds are formed between reactants). Also as stated above, the Arrhenius equation provides that as a rough rule of thumb, reaction rates for many reactions double or triple for every 10 degrees Celsius increase in temperature.

In many cases, a 20-40 degree increase (over room temperature) may be achieved; e.g., up to about 130 or about 150 degrees F. This might result in the minimum of about 10 degrees C. desirable to double or triple the chemical cleaning reactions. This may be so even at about a half inch or more above the heating element.

In some alternatives, the bristles may have heat conductive properties, as in perhaps having some metallic or other conductive parts disposed or formed therein. Indeed, in some implementations, the bristles may be or form the heating element or a part thereof. In some cases, the bristles may be a metallic bristle pad. Note also that the bristles may, in some implementations, be formed or tufted right in the heating element, whether heat conductive or otherwise.

Note, the implementations described thus far may involve current flowing through the heating element, and thus through conductive components in contact therewith. Thus, it may be that some such elements could come into contact with living tissue; however, the current levels are expected to be so small that no harm would come to any living tissue coming into contact therewith.

An alternative implementation may include an induction heating element which might involve a circuit with, for example, an induction coil operatively disposed adjacent to a heating element to be heated. In such case, the heating element may be of a different material and shape, so as to have a sufficient induction current formable therein by the induction coil, or like element. The heating element would then pass the heat to the operative oral area, substantially as described above.

Also considered are heated tooth whitening trays which when disposed in the mouth with a tooth whitening composition therein may also enhance and/or speed the chemical reactions and thus the process time thereof. For example, peroxide or other whiteners may work faster in elevated temperatures for reasons such as those described above. Similarly, a dental polisher may be heated in this way and speed a polishing procedure. In this way, a large variety, if not all, oral care devices which may have reason to be introduced in the oral cavity may include heating as described here.

Other alternatives within the scope hereof are included as well.

Accordingly, what is claimed is:

1. A temperature modified oral care device comprising:
a base;
an electrical circuit disposed at least partially in said base; and,
a heating element connected to the electrical circuit.

2. A temperature modified oral care device according to claim 1, wherein the temperature modified oral care device is a toothbrush.

3. A temperature modified oral care device according to claim 1, wherein the temperature modified oral care device is a tooth polisher.

4. A temperature modified oral care device according to claim 1, wherein the temperature modified oral care device is a tooth whitening tray.

5. A temperature modified oral care device according to claim 1 wherein the heating element is selected from the group consisting of graphite, a composition containing graphite, athalite or a combination of the foregoing.

6. A temperature modified oral care device according to claim 1 wherein the heating element is one or more of germanium or silicon.

7. A temperature modified oral care device according to claim 1 wherein said device is rapid heating.

8. A temperature modified oral care device according to claim 1 wherein said device is rapid cooling.

9. A temperature modified oral care device according to claim 1, wherein the toothbrush has temperature modified bristles.

10. A temperature modified oral care device according to claim 1, wherein the toothbrush has one or more of a safety activation button.

11. A temperature modified oral care device according to claim 1, wherein the toothbrush has a glowing indicator to indicate activation of a heating element.

12. A system for using a temperature modified oral care device, the system comprising:
a temperature modified oral care device; and
a toothpaste, gel, or dentifrice, to be disposed upon the temperature modified oral care device.

13. A method of using of a temperature modified oral care device, comprising applying system according to claim 12 to an oral cavity, and cleaning at least a portion of the oral cavity.

14. A method for using a temperature modified oral care device, the method comprising:
activating a temperature modified oral care device by engaging an electrical circuit, wherein the electrical circuit is disposed within the oral care device and modifies the temperature of the oral care device;
placing the temperature modified oral care device in the oral cavity; and
engaging said oral care device.

15. A method for using a temperature modified oral care device according to claim 14, the method comprising the additional step of applying a toothpaste, gel, or other dentifrice to the oral care device.
